(19) United States
(12) Patent Application Publication
(10) Pub. No.: US 2024/0350309 A1
(43) Pub. Date: Oct. 24, 2024

(54) CONSOLE AND CASSETTE FOR AN OPHTHALMOSURGICAL SYSTEM, AND OPHTHALMOSURGICAL SYSTEM

(71) Applicant: CARL ZEISS MEDITEC AG, Jena (DE)

(72) Inventors: Andreas WERNER, Aalen (DE); Rupert DEMLEITNER, Heidenheim (DE); Peter EICHERT, Bissingen (DE); Steffen KIBBEL, Lauchheim (DE)

(21) Appl. No.: 18/687,971

(22) PCT Filed: Sep. 13, 2022
(86) PCT No.: PCT/EP2022/075376

§ 371 Date: Feb. 29, 2024

(30) Foreign Application Priority Data
Sep. 21, 2021 (DE) 10 2021 210 484.3

(51) Int. Cl. A61F 9/007; A61M 1/00

(52) U.S. Cl. CPC A61F 9/00736; A61M 1/77; A61M 2205/121; A61M 2205/14; A61M 2210/0612

(57) ABSTRACT

Disclosed is a console for an ophthalmosurgical system, having a cassette holder into which a cassette is insertable for controlling the flow of an irrigation fluid to a surgical instrument for an eye that is to be treated, and having a clamping device assigned to the cassette holder, for releasably securing a cassette to the console. The clamping device is adjustable between an open position, which permits insertion of a cassette into the cassette holder and removal of a cassette from the cassette holder, a positioning position, in which a cassette is at least partially engaged around by a pivoting of the clamping device and is positioned in the cassette holder, and a clamping position, in which a cassette is secured on the cassette holder by a linear adjustment of the clamping device. Also disclosed are an associated cassette, and ophthalmosurgical system.

The invention relates to a console according to the preamble of claim 1. The invention also relates to a cassette for an ophthalmosurgical system, and to an ophthalmosurgical system.

There are a number of surgical techniques for treating clouding of the crystalline lens, which is referred to in medicine as a cataract. The most widespread technique is phacoemulsification, in which a thin hollow needle is introduced into the crystalline lens and is induced to make ultrasonic vibrations. In its immediate surroundings, the vibrating hollow needle emulsifies the lens in such a way that the resulting lens particles can be aspirated through a line by means of a pump. A flushing fluid (irrigation fluid) is delivered during this process, with the aspiration of the lens particles and of the fluid taking place through an aspiration line. When the lens has been completely emulsified and removed, a new artificial lens can be inserted into the empty capsular bag, and so a patient treated in this way can recover good vision.

The ophthalmosurgical system normally used in these cases usually comprises a console to which an irrigation fluid container can be coupled, with an irrigation fluid contained therein. This can be done, for example, by means of a stand with a hook on which the irrigation fluid container can be suspended. In addition, the ophthalmosurgical system has a cassette, which is inserted into the console. The cassette serves to steer the irrigation fluid to a surgical instrument for the eye that is to be treated. The surgical instrument is used to crush a lens of the eye, for example by phacoemulsification, it being possible to aspirate the resulting lens particles and the irrigation fluid using the cassette. In this case, the irrigation fluid is first pumped from the irrigation fluid container to the cassette, from there to the surgical instrument, and finally back into the cassette.

The precise and defined positioning and attachment of the cassette to the console is critical in particular. Thus, it must be ensured that the cassette is connected to the console firmly and in a stable position during operation, in order to ensure smooth operation and corresponding accuracy. However, since the cassette is a sterile disposable product that is typically produced from injection-molded components, it is important at the same time to ensure that the cassette is subjected to the least possible mechanical stress when it is received in the console. Only then can the cassette be produced using the least amount of material and thus in a cost-effective manner. It must also be ensured that the cassette can be connected to the non-sterile console under sterile conditions by a sterile person, without the risk of the sterile person being made non-sterile. In addition, it must also be possible for the cassette to be exchanged as easily as possible in order not to adversely affect the operation sequence and to keep the turn-over times between two operations as short as possible.

DE 603 03 057 T2 discloses a console in which the cassette is clamped via two pivot arms, which for this purpose are pivoted by a motor. However, it has proven disadvantageous in DE 603 03 057 T2 that the adjustment of the pivot arms requires a very complex design. Moreover, in the solution known from the prior art, it has been found to be problematic that the pivoting movement for clamping the cassette increases the risk of damage to the cassette, or that the cassette accordingly has to be made robust, which has a detrimental effect on the production costs.

The object of the present invention is to reduce the disadvantages outlined above and to make available an improved console. In addition, the object of the invention is to make available an improved cassette and an improved ophthalmosurgical system.

According to the invention, the object concerning the console is achieved, in the case of a console according to the preamble of claim 1, by the fact that the clamping device is adjustable between an open position, which permits insertion of a cassette into the cassette holder and removal of a cassette from the cassette holder, a positioning position, in which a cassette is at least partially engaged around by a pivoting of the clamping device and is positioned in the cassette holder, and a clamping position, in which a cassette is secured on the cassette holder by a linear adjustment of the clamping device.

By virtue of the different movement of the clamping device—the pivoting movement for positioning the cassette, and the linear adjustment of the clamping device for actually clamping the cassette-a clear separation is obtained between the two positions “positioning position” and “clamping position”. In addition, it can thus be ensured that, when the cassette is actually clamped on the cassette holder, the force acting on the cassette can be dosed very precisely and, by the parallel adjustment of the clamping device, the contact region between the clamping device and the cassette also always remains the same. Moreover, through the division of the movements, it is possible to reduce the risk of the user accidentally catching a finger or other part of the body in the clamping device, when the clamping device is moved from the positioning position to the clamping position.

It has also proven expedient in particular if at least one actuator is provided for the linear adjustment of the clamping device. In particular, by the activation of the actuator, the clamping device is moved linearly from the positioning position to the clamping position against the cassette holder, as a result of which a cassette located in the cassette holder is clamped in the latter. In this case, it has proven to be particularly advantageous if the actuator is pneumatically adjustable and is preferably formed as a cylinder having a piston which can be acted on by pressure medium, such as compressed air or hydraulic fluid. In this case, it is also provided in particular that only one actuator is provided for the clamping device. Within the scope of the invention, however, the actuator can also be provided in a plurality thereof and/or as an electric motor. In a particularly preferred embodiment, the actuator is adjusted from the positioning position to the clamping position counter to the force of a return element. If the actuator is deactivated, i.e. depressurized or de-energized, the clamping device is automatically transferred from the locked position back to the positioning position by the return element. In other words, the clamping device is moved away from the cassette holder again by the return element, which can be achieved, for example, by one or more pretensioned springs.

In this context, it has also proven expedient if, for triggering the adjustment of the clamping device from the positioning position to the clamping position, at least one sensor is provided that detects a cassette inserted in the cassette holder. Thus, if a cassette is inserted into the cassette holder, the cassette is detected by the sensor, which is preferably formed as a distance sensor, for example as an inductive proximity sensor, and only then is the adjustment of the actuator activated, in order to move the clamping device linearly from the positioning position to the clamping position.

It has also proven expedient if the clamping device comprises at least two clamping hooks, which are each pivotable from the open position to the positioning position counter to the force of a return spring. The clamping hooks, which are particularly preferably oriented substantially vertically, make it particularly easy to clamp the cassette in the cassette holder and to achieve improved stiffness of the cassette, especially when the cassette is inserted edgeways into the cassette holder. Given the course of the fluidic channels within the cassette, the stiffness of the cassette in the transverse direction, which runs horizontally in the insertion state, is in fact greater than in the longitudinal direction, which runs vertically in the insertion state. In addition, the vertical clamping of the cassette, compared to clamping of the cassette at its top and bottom, allows a shorter length of the cassette, which likewise increases the stiffness of the cassette. In addition, the return springs ensure that the clamping hooks and thus the clamping device can be moved from the positioning position back to the open position when the cassette is to be removed from the cassette holder. Within the scope of the invention, however, it is also provided that the cassette is not clamped, as in the present case, at its longitudinal sides, but at its transverse sides. In addition, it is also provided within the scope of the invention that the longitudinal sides are horizontal and the transverse sides of the cassette are vertical.

It has also proven advantageous if the clamping hooks are each assigned a lever arm, which is arranged in such a way that, upon insertion of the cassette into the cassette holder, a pivoting of the clamping hook is effected counter to the force of the return spring. When the cassette is inserted, the lever arms of the clamping hooks are acted upon by the cassette, as a result of which the clamping device is pivoted about the pivot axis and automatically transferred from the open position to the positioning position. In the positioning position, the clamping hooks at least partially engage around the cassette, which in a simple way prevents the inserted cassette from tipping out of the cassette holder.

It has also been shown to be expedient if the clamping hook is connected to a pull rod via a securing pin, and if the securing pin forms the pivot axis of the clamping hook. This makes it easy to pivot the clamping device between the open position and the positioning position. In addition, for the linear adjustment of the clamping device from the positioning position to the clamping position, the pull rod simply has to be pulled.

In order to pretension the clamping device, it has also proven particularly expedient if the clamping hook is assigned a wedge surface, which pretensions the return spring during the pivoting of the clamping device from the open position to the positioning position. This then assists in particular the ejection of the cassette from the cassette holder. In this case, it is provided in particular that an adjustment sleeve is used, which is acted on by the wedge surface and is adjusted counter to the force of the return spring.

In this context, it has also been shown to be advantageous if a retaining bracket is provided, which is preferably oriented perpendicular to the clamping device. The retaining bracket provides additional securing of the cassette. The retaining bracket is preferably arranged, relative to a base surface of the console, at the upper region of the cassette holder. This means ultimately that the retaining bracket, mounted at the top, prevents the cassette from accidentally tipping out. In this case, it is particularly preferred if the retaining bracket is spring-loaded. The spring loading and the correspondingly shaped insertion bevels on the retaining bracket and/or on the cassette have the effect that, when the cassette is inserted manually, the retaining bracket can firstly deflect upward or swing out counter to the spring force and then snap over the inserted cassette, thereby additionally securing the latter in the cassette holder. Moreover, the retaining bracket makes it easier to release the cassette received in the cassette holder. When the clamping device is located in the positioning position, i.e. the clamping device is pivoted inward but not linearly moved against the console, the user simply has to lift the retaining bracket counter to the spring, as a result of which the clamping hooks, supported by the return springs, are pivoted out again, which leads to ejection of the cassette, which can then be removed from the cassette holder by the user.

It has also proven expedient if the retaining bracket is arranged in such a way that an adjustment of the retaining bracket actuates a switch that switches the actuator. In particular, in the clamping position, that is to say with the cassette clamped, the adjustment of the retaining bracket can actuate the switch, which switches the actuator such that the clamping device is transferred from the clamping position to the positioning position. Alternatively, however, the user can also actuate the actuator using an operator interface, also known as a GUI, that is designed on the console. As has been described above, however, the retaining bracket is also adjusted upon insertion of the cassette, as a result of which the switch is actuated and thus the actuator is switched in order to move the clamping device from the positioning position to the clamping position, which, in a particularly preferred embodiment, is possible only when the presence of a cassette in the cassette holder is detected by the sensors.

To make it easier to attach the cassette to the cassette holder, it has been shown to be advantageous if guide rails are assigned to the cassette holder. The guide rails, which are preferably formed parallel to the clamping device, are preferably short and extend, according to the invention, over less than 20%, preferably over less than 15%, and more preferably over less than 10% of the length of the cassette. This allows the cassette to be inserted into the cassette holder obliquely from above.

To allow a defined orientation of the cassette, it has also proven advantageous if at least two alignment pins are assigned to the cassette holder for the purpose of aligning the cassette. These alignment pins, which engage in the cassette, can ensure a defined fit of the cassette, especially during the insertion of the cassette. The two alignment pins, and the flat surface of the cassette holder against which the cassette is pressed in the clamping position, will ensure a spatially defined positioning of the cassette, i.e. fixing of the cassette in all three translational and rotational spatial directions. In this context, it has also proven advantageous if at least one of the alignment pins is arranged offset with respect to the longitudinal axis of the cassette holder. The longitudinal axis is oriented vertically.

According to the invention, the object concerning the cassette is achieved by a cassette to be received in a console of an ophthalmosurgical system, in which a slot-shaped recess and a receptacle are formed in the cassette for the purpose of receiving alignment pins of a cassette holder of a console of the ophthalmosurgical system. The slot-shaped recess allows the cassette to be initially placed at an angle on one of the alignment pins and then manually tilted in the direction of the cassette holder, the other alignment pin then engaging in the receptacle of the cassette, as a result of which the cassette is aligned with the cassette holder. In particular, the slot-shaped recess has proven particularly positive since, for example when using a further receptacle instead of the slot-shaped recess, the system would otherwise be statically overdetermined.

In order to simplify the production of the cassette, it has also proven expedient if the slot-shaped recess and/or the first receptacle are at least partially formed on all the housing parts of the cassette. This makes it possible for the individual parts of the cassette, which are connected to one another by laser welding, to be oriented relative to one another before the actual joining process. For this purpose, the dimensions of the recess, formed in the individual housing parts of the cassette, and of the receptacle are designed and coordinated in such a way that any manufacturing deviations that occur can be minimized and thus the positioning of the components with respect to one another can be optimized. For aligning the housing parts of the cassette, it is possible to use the same alignment pins also formed on the cassette holder.

Typically, the individual housing parts of the cassette are successively interconnected until the cassette has its final shape.

According to the invention, the object concerning the ophthalmosurgical system is achieved by an ophthalmosurgical system having a console according to any one of claims 1 to 13, and having a cassette according to either of claims 14 and 15.

The invention is explained in more detail below on the basis of a number of exemplary embodiments illustrated in the drawings, in which:

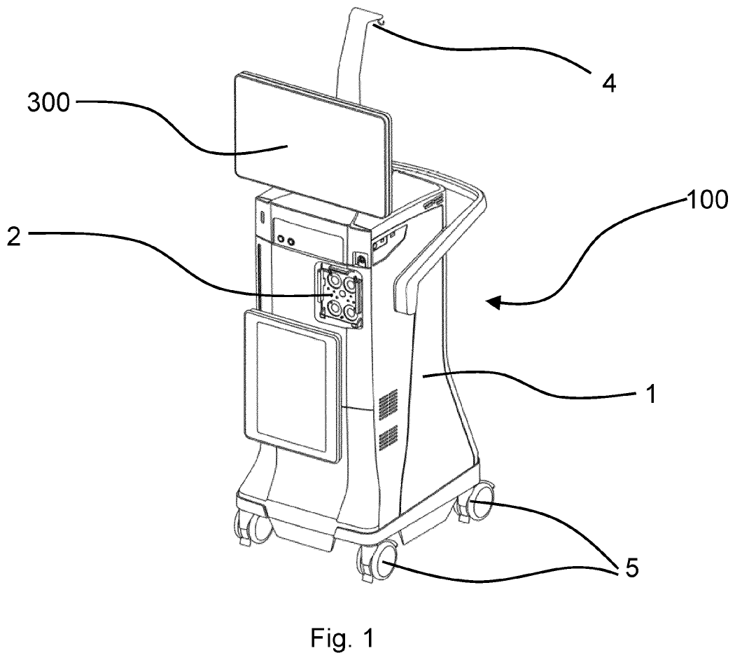

FIG. 1 shows a perspective view of a console,

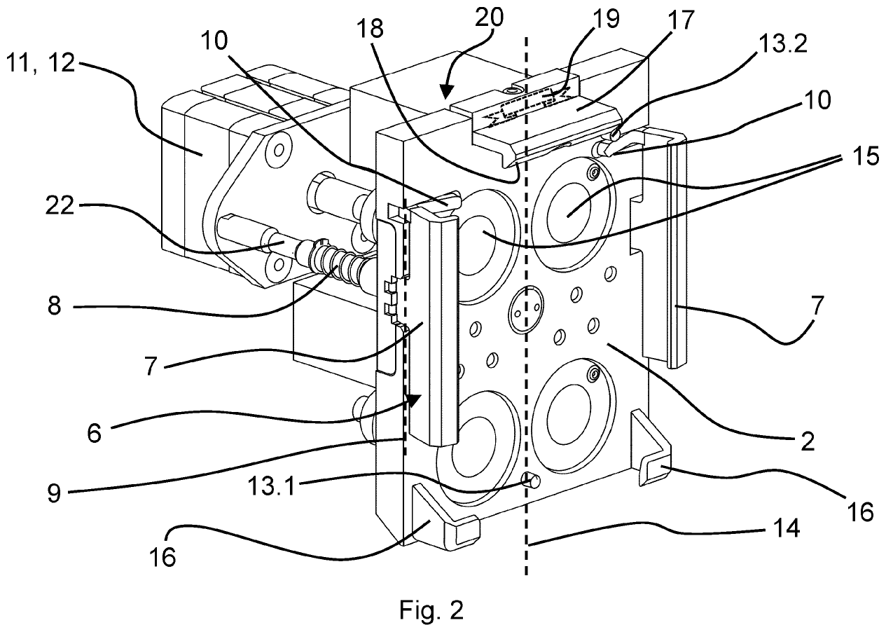

FIG. 2 shows a detailed perspective view of a cassette holder of the console, with a clamping device in an open position,

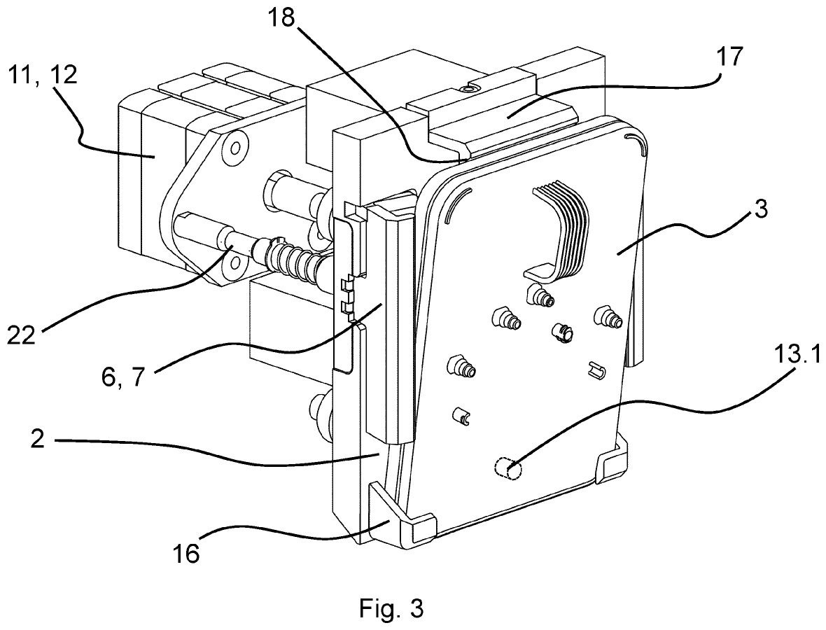

FIG. 3 shows a detailed perspective view of the cassette holder, with an attached cassette,

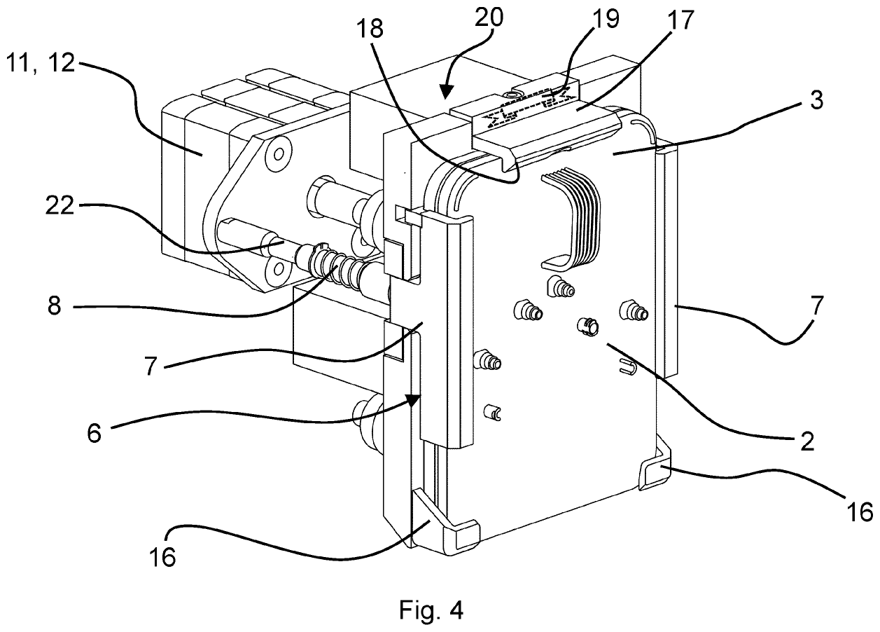

FIG. 4 shows a detailed perspective view of a cassette, received in the cassette holder of the console, with the clamping device in a positioning position,

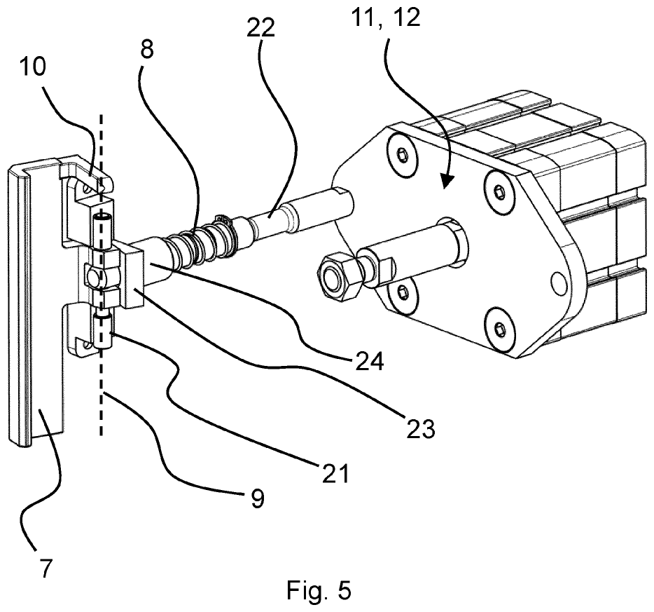

FIG. 5 shows a detailed view of the clamping device,

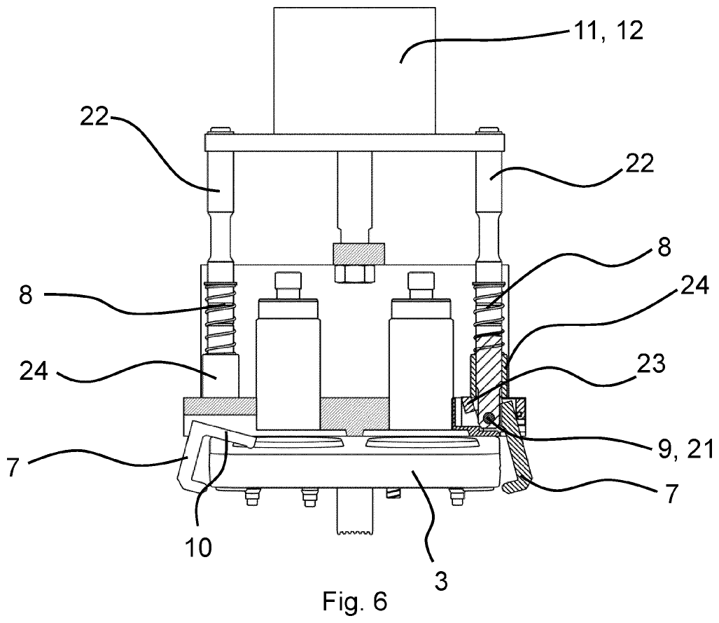

FIG. 6 shows a partially sectional view of the cassette holder, and

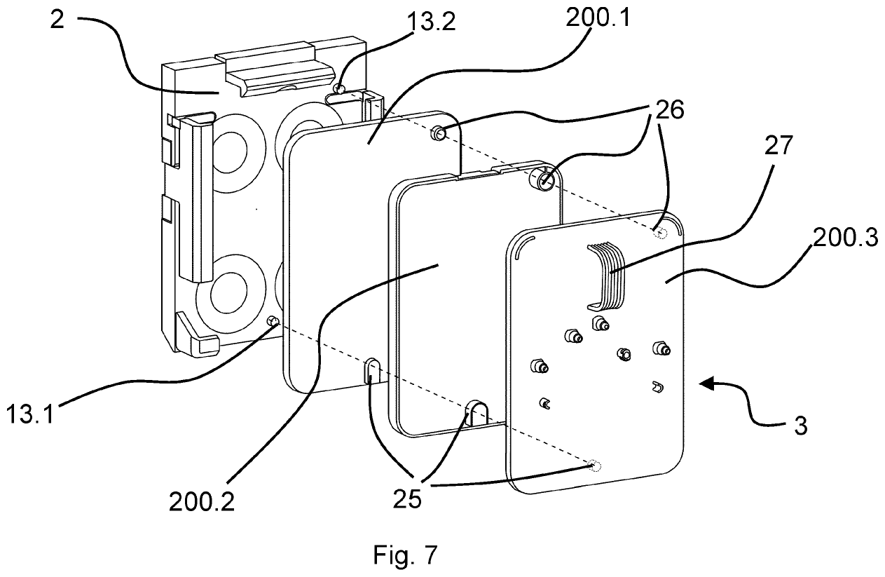

FIG. 7 shows an exploded view of the cassette.

FIG. 1 shows a perspective view of a console 1 of an ophthalmosurgical system 100. On the front of the console 1, a cassette holder 2 is formed into which a cassette 3 is insertable for controlling the flow of an irrigation fluid to a surgical instrument (not shown in the drawing) for an eye that is to be treated. The irrigation fluid container holding the irrigation fluid, usually a bottle, can be suspended on a stand 4, which is mounted on the console 1. To be able to adjust the position of the console 1 during the operation, the console 1 is assigned wheels 5, with which the console 1 can be moved. As will be explained in more detail below with reference to FIGS. 2 to 4 in particular, the console 1 has a clamping device 6, which is assigned to the cassette holder 2. With the aid of this clamping device 6, the cassette 3 can be secured releasably to the console 1. In addition, the console 1 is assigned a user interface 300, also referred to as a graphical user interface (GUI).

The exact procedure of inserting the cassette 3 into the cassette holder 2 and of removing it from the cassette holder 2 is explained in detail below with reference to FIGS. 2 to 4. FIG. 2 shows the cassette holder 2 before insertion of the cassette 3 into the cassette holder 2, with the clamping device 6 in the open position. FIG. 3 shows the actual procedure of inserting the cassette 3 into the cassette holder 2, and FIG. 4 lastly shows the clamping device 6 in the positioning position, in which the clamping device 6 is pivoted over the cassette 3.

In the open position shown in FIG. 2, insertion of the cassette 3 into the cassette holder 2 and removal of a cassette 3 from the cassette holder 2 by the user is possible. In the exemplary embodiment shown, the clamping device 6 comprises two clamping hooks 7, which are each pivotable counter to the force of a return spring 8 about a pivot axis 9 from the open position shown in FIG. 2 to a positioning position shown in FIG. 4. In the open position, the clamping hooks 7 of the clamping device 6 are adjusted outward, such that the clamping hooks 7 do not impede insertion of the cassette 3 into the cassette holder 2. In the positioning position, the cassette 3 is then at least partially engaged around by a pivoting movement of the clamping device 6, or of the clamping hooks 7, and positioned in the cassette holder 2. As can be seen from FIG. 2, the clamping hooks 7 are oriented substantially vertically, and the cassette 3 is enclosed at their vertically extending longitudinal sides. As is explained in detail below again with reference to FIGS. 5 and 6, the clamping hooks 7 are each assigned a lever arm 10. Upon insertion of the cassette 3 into the cassette holder 2, this lever arm 10 is acted on by the cassette 3 and causes the pivoting of the corresponding clamping hook 7 from the open position to the positioning position counter to the force of the return spring 8.

Moreover, the console 1 also comprises an actuator 11, with which it is possible to adjust the clamping hooks 7 linearly from the positioning position in the direction of the cassette holder 2 into a clamping position, in which the cassette 3 is secured on the cassette holder 2 by a clamping action. The actuator 11 is designed as a cylinder 12, the piston of which can be subjected to a pressure medium in order to pull the clamping hooks 7 from the positioning position to the clamping position and thus in the direction of the cassette holder 2, as a result of which the cassette 3 is ultimately clamped on the cassette holder 2.

It will also be seen from FIG. 2 that two alignment pins 13.1, 13.2 are arranged on the cassette holder 2, of which a first alignment pin 13.1 lies on a longitudinal axis 14 of the cassette holder 2, while a second alignment pin 13.2 is spaced apart from the longitudinal axis 14. The alignment pins 13.1, 13.2 serve to precisely position the cassette 3 on the cassette holder 2. In addition, in the exemplary embodiment shown, two sensors 15 are arranged in the cassette holder 2, which sensors detect when the cassette 3 is inserted into the cassette holder 2. In this case, the sensors 15, which are preferably designed as inductive proximity sensors, are triggered only when the cassette 3 is in the immediate vicinity of the sensors 15, i.e. when the clamping device 6 is already in the positioning position. When the cassette 3 is detected in the positioning position by the sensors 15, the actuator 11 can be activated, as a result of which the clamping device 6 is adjusted linearly from the positioning position to the clamping position.

As is shown in FIG. 3, upon insertion of the cassette 3 into the cassette holder 2, the cassette 3 is first placed at an angle onto the first alignment pin 13.1, which is located on the longitudinal axis 14 of the cassette holder 2 and which is indicated in FIG. 3 only with a dashed line. Placing the cassette 3 onto the first alignment pin 13.1 is made easier by guide rails 16, which are formed on the cassette holder 2, and of which the length is less than 10% of the length of the cassette 3 in the exemplary embodiment shown. If the user now presses the cassette 3 further into the cassette holder 2, the cassette 3 comes to bear on a retaining bracket 17, which is oriented perpendicular to the clamping device 6 and is spring-loaded. By means of an insertion bevel 18 formed on the retaining bracket 17, the retaining bracket 17 is deflected counter to the force of a spring 19 and latches over the cassette 3 inserted into the cassette holder 2. At the same time, the cassette 3 acts on the lever arms 10 of the clamping hooks 7 during insertion, as a result of which the clamping hooks are transferred from the open position to the positioning position, i.e. they pivot inward and engage at least partially around the cassette 3. The clamping device 6 is now located in the positioning position shown in FIG. 4.

Here, the two alignment pins 13.1, 13.2 engage in the cassette 3, as will be explained below with reference to FIG. 7. When adjusting the retaining bracket 17, a switch 20 is also actuated, which switches the actuator 11. Since the cassette 3 is now located very close to the sensors 15, the presence of the cassette 3 is detected, such that the actuator 11 is switched on and the clamping device 6 is adjusted linearly in the direction of the cassette holder 2 into the clamping position. In the exemplary embodiment shown, the sensors 15, and also the switch 20 actuated by the retaining bracket 17, are ultimately necessary for activating the actuator 11 and for adjusting the clamping device 6 from the positioning position to the clamping position. If the cassette 3 is now to be removed again from the cassette holder 2, the retaining bracket 17 simply needs to be opened manually, as a result of which the switch 20 that switches the actuator 11 is first of all actuated and deactivates the actuator 11. Since the actuator 11, and with it the clamping device 6, is pretensioned in the clamping position by the force of a return element not shown in the drawing, the clamping device 6 is transferred away from the cassette holder 2 into the positioning position when the actuator 11 is switched off. Since the clamping hooks 7 are pretensioned by the return springs 8, the clamping device 6 is now automatically transferred from the positioning position to the open position, that is to say the clamping hooks 7 pivot outward and thus release the cassette 3, and the cassette 3 is ejected from the cassette holder 2 by the lever arms 10 of the clamping hooks 7 and can be removed from the console 1 by the user. However, the ejection of the cassette 3 from the cassette holder 2 can also be initiated by actuating the user interface 300. In the exemplary embodiment shown, the actuator 11 is designed such that, even in the de-energized and/or depressurized state, the clamping device 6 is transferred from the clamping position to the positioning position by the elastic return element, when the retaining bracket 17 is opened manually. This at all times ensures that the cassette 3 can be removed from the console 1, even when the console 1 is deactivated.

The mechanical operation of the adjustment of the clamping device 6 between the open position, the positioning position and the clamping position is explained in more detail below with reference to FIGS. 5 and 6. From the detailed view of one of the clamping hooks 7 of the clamping device 6 shown in FIG. 5, and in particular from the partially sectional view of the clamping device 6 shown in FIG. 6, it is clear that the clamping hook 7 is connected pivotably, but axially rigidly, to a pull rod 22 via a securing pin 21, the securing pin 21 at the same time forming the pivot axis 9 of the clamping hook 7. When the cassette 3 is placed into the cassette holder 2, the cassette 3 acts on the lever arms 10 formed on the clamping hooks 7, as a result of which the clamping hooks 7 are pivoted about the pivot axes 9. In this case, a wedge surface 23 connected to the clamping hook 7 is also pivoted in each case, as a result of which an adjustment sleeve 24 is adjusted counter to the force of the return spring 8 and pretensions the latter. If the actuator 11 is now switched on, the pull rods 22 and the clamping hooks 7 connected to the pull rods 22 via the securing pin 21 are moved linearly to the clamping position, as a result of which the cassette 3 located in the cassette holder 2 is clamped therein.

FIG. 7 shows a cassette 3 and the cassette holder 2 in an exploded view. FIG. 7 shows clearly that a slot-shaped recess 25 and at least one receptacle 26 are formed in the cassette 3 for the purpose of receiving the first alignment pin 13.1 and the second alignment pin 13.2 of the cassette holder 2 of the console 1 of the ophthalmosurgical system 100, as a result of which the cassette 3 is aligned with the cassette holder 2, as is indicated in FIG. 7 by the dashed lines. The slot-shaped recess 25 and the receptacle 26 are at least partially formed on all three housing parts 200.1, 200.2, 200.3 of the cassette 3 and facilitate the process of producing the cassette 3, since the slot-shaped recesses 25 and the receptacles 26, which are formed in the individual housing parts 200.1, 200.2 and 200.3, mesh during assembly and allow the components to align with one another. It can also be seen from FIG. 7 that the cassette is assigned a handle 27 into which the user can place his or her index finger, for example, in order to insert the cassette 3 into the cassette holder 2 and to remove it again from the latter.

### LIST OF REFERENCE SIGNS

1 console
2 cassette holder
3 cassette
4 stand
5 wheel
6 clamping device
7 clamping hook
8 return spring
9 pivot axis
10 lever arm
11 actuator
12 cylinder
13.1 first alignment pin
13.2 second alignment pin
14 longitudinal axis of cassette holder
15 sensor
16 guide rail
17 retaining bracket
18 insertion bevel
19 spring
20 switch
21 securing pin
22 pull rod
23 wedge surface
24 adjustment sleeve
25 slot-shaped recess
26 receptacle
27 handle
100 ophthalmosurgical system
200.1 housing part
200.2 housing part
200.3 housing part
300 user interface

1. A console for an ophthalmosurgical system, comprising:
a cassette holder into which a cassette is insertable for controlling the flow of an irrigation fluid to a surgical instrument for an eye that is to be treated, and
a clamping device assigned to the cassette holder, for releasably securing a cassette to the console,
wherein the clamping device is adjustable between an open position, which permits insertion of a cassette into the cassette holder and removal of a cassette from the cassette holder, a positioning position, in which a cassette is at least partially engaged around by a pivoting of the clamping device and is positioned in the cassette holder, and a clamping position, in which a cassette is secured on the cassette holder by a linear adjustment of the clamping device.

2. The console as claimed in claim 1, wherein at least one actuator is provided for the linear adjustment of the clamping device.

3. The console as claimed in claim 1, wherein, for triggering the adjustment of the clamping device from the positioning position to the clamping position, at least one sensor is provided that detects a cassette inserted in the cassette holder.

4. The console as claimed in claim 1, wherein the clamping device comprises at least two clamping hooks, which are each pivotable from the open position to the positioning position counter to the force of a return spring.

5. The console as claimed in claim 4, wherein the clamping hooks are oriented substantially vertically.

6. The console as claimed in claim 4, wherein the clamping hooks are each assigned a lever arm, which is arranged in such a way that, upon insertion of the cassette into the cassette holder, a pivoting of the clamping hook is effected counter to the force of the return spring.

7. The console as claimed in claim 4, wherein the clamping hook is connected to a pull rod via a securing pin, the securing pin forming the pivot axis of the clamping hook.

8. The console as claimed in claim 4, wherein the clamping hook is assigned a wedge surface, which pretensions the return spring during the pivoting of the clamping device from the open position to the positioning position.

9. The console as claimed in claim 1, wherein a retaining bracket is provided, which is preferably oriented perpendicular to the clamping device.

10. The console as claimed in claim 9, wherein the retaining bracket is arranged in such a way that an adjustment of the retaining bracket actuates a switch that switches the actuator.

11. The console as claimed in claim 1, wherein guide rails are assigned to the cassette holder.

12. The console as claimed in claim 1, wherein at least two alignment pins are assigned to the cassette holder for the purpose of aligning the cassette.

13. The console as claimed in claim 12, wherein at least one of the alignment pins is arranged offset with respect to the longitudinal axis of the cassette holder.

14. A cassette to be received in the console of an as claimed in claim 1, wherein a slot-shaped recess and at least one receptacle are formed in the cassette for the purpose of receiving alignment pins of the cassette holder of the console.

15. The cassette as claimed in claim 14, wherein the slot-shaped recess and/or the at least one receptacle are at least partially formed on all the housing parts of the cassette.

16. An ophthalmosurgical system having the console as claimed in claim 1, and having a cassette to be received in the console, wherein a slot-shaped recess and at least one receptacle are formed in the cassette for the purpose of receiving alignment pins of the cassette holder of the console.
